(19) United States
(12) Patent Application Publication
(10) Pub. No.: US 2011/0183352 A1
(43) Pub. Date: Jul. 28, 2011

(54) Configurable Diagnostic Systems and Methods for Performing Assays

(76) Inventors: Emmanuel C. Mpock, Salida, CA (US); Wilma Mangan, Santa Clara, CA (US)

(21) Appl. No.: 12/692,445

(22) Filed: Jan. 22, 2010

(51) Int. Cl. G01N 33/53

(52) U.S. Cl. 435/7.1; 435/287.2; 422/400

(57) ABSTRACT

A method and system for configuring an analyzer is disclosed. The analyzer receives a strip identifier from a strip or a vial identifier from a vial. The parameter module in the analyzer determines the parameters corresponding to the received strip identifier or the vial identifier. The parameter module then configures the analyzer to perform a test with the strip using the determined parameters. In one embodiment, the diagnostic test module determines the test corresponding to the received strip identifier or the vial identifier and the diagnostic test module configures the analyzer to perform the determined test with the strip. In another embodiment, the association determination module determines if the received strip identifier and vial identifier are associated with each other. If not, the analyzer renders an error requesting a correct strip.

### FIELD OF INVENTION

The present invention relates to assay systems and more particularly to configurable systems for performing assays.

### BACKGROUND

Qualitative and quantitative immuno- and chemical assays have gained acceptance as important tools in the medical and food industries. These methods have been used for the diagnosis of disease conditions, detection of analytes, and for the detection of microbes, such as bacteria. These methods of diagnosis have established effectiveness, and the methods have made it easier for physicians to monitor and manage patients undergoing various forms of therapy.

Traditionally, the diagnostic assays have been performed in hospital and clinic settings, and involve the use of sophisticated and expensive equipment, that require specially trained personnel for their operation. Further, the assay results are sometimes not available for days or weeks after the samples from the patients have been obtained. The presently available diagnostic assays are thus costly, time consuming, and not convenient.

Attempts have been made to develop less costly assays. For example, a typical home self-test for detecting blood components requires the patient to prick a finger with a sterilized lancet, apply a drop of blood sample to a sample application area on the disposable strip, and then wait for the results. Assays that use other bodily fluids, such as urine essentially work in a similar manner. These devices are designed such that a typical lay person can perform the assays correctly with very little training. However, these assay systems generally suffer from low accuracy or require a number of preparative steps be performed that could compromise the test results, and are thus not convenient.

One of the preparative steps involves configuring the assay system for a particular test or a particular type of test strip. If the assay is not configured properly, the results may be inaccurate if not misleading. Manual configuration is prone to human error and adds another hurdle that the patient or a caretaker has to pass before the patient can be tested.

### SUMMARY

The present invention provides systems and methods for configuring an analyzer that performs diagnostic test using one of the plurality of test strips from a vial. The strip comprises a strip information module that stores information about the strip like an identifier associated with the strip. In one embodiment, the strip information module includes information about the vial associated with the strip. In another embodiment, the strip information module includes a diagnostic test or an identifier for the diagnostic test that should be performed with the strip. In another embodiment, the strip information module includes parameters used to configure the diagnostic test to be performed by the strip.

The vial comprises a vial information module that stores information about the vial like an identifier associated with the vial. In one embodiment, the vial identifier module includes information about the strips associated with the vial. In another embodiment, the vial information module includes a diagnostic test or an identifier for the diagnostic test that should be performed with one of the strips associated with the vial. In another embodiment, the vial information module includes parameters used to configure the diagnostic test to be performed with the associated strip.

The analyzer comprises a diagnostic controller, a diagnostic test module, an association determination module, a parameter module and a storage. The diagnostic controller in the analyzer receives information from the strip or the vial and directs one of the other modules in the analyzer to perform a particular function. For example, the diagnostic controller is configured to direct the association determination module to determine whether a strip is associated with a vial. The diagnostic controller is also configured to direct the diagnostic test module to configure the analyzer with a test associated with a received strip identifier or vial identifier. Moreover, the diagnostic controller is configured to direct the parameter module to configure the analyzer for using parameter in a test corresponding to the received strip identifier and/or vial identifier.

In one embodiment, the diagnostic controller receives a strip identifier from the strip and a vial identifier from the vial. The diagnostic controller then directs the association determination module to determine whether the received strip identifier and the vial identifier are associated with each other. The association determination module queries the storage in analyzer to determine the vial identifiers associated with the received strip identifier or determine the strip identifiers associated with the received vial identifier. If the received strip identifier is one of the associated strip identifiers retrieved from storage or if the received vial identifier is one of the associated vial identifier retrieved from storage, then the association determination module determines that the received strip identifier and vial identifiers are associated. The association determination module transmits the results of its determination to the diagnostic controller.

In another embodiment, the diagnostic controller directs the diagnostic test module to determine a test associated with the received strip identifier or vial identifier. The diagnostic test module retrieves a test corresponding to the received strip identifier or the vial identifier and configures the analyzer to perform the retrieved test with the strip. In one embodiment, the diagnostic controller receives a test identifier or the test itself from the strip or the vial and transmits the received test identifier or the test to the diagnostic test module. The diagnostic test module then configures the analyzer with the received test. If the diagnostic test module receives a test identifier, the diagnostic test module retrieves from storage a test corresponding to the received test identifier and configures the analyzer with the retrieved test.

In yet another embodiment, the diagnostic controller directs the parameter module to configure the analyzer to perform a test using parameters corresponding to the received strip identifier or vial identifier. The parameter module retrieves from storage one or more parameters corresponding to the received strip identifier or the vial identifier and configures the analyzer to perform a test using the retrieved parameters.

### BRIEF DESCRIPTION OF DRAWINGS

FIG. 1 illustrates a base system and a disposable strip according to one embodiment of the invention.

FIG. 2 illustrates a perspective view of the disposable strip with a set of interconnected reaction chambers and a moveable member in one of the chambers according to one embodiment of the invention.

FIG. 3 illustrates another view of the disposable strip comprising a composite of membranes assembled to isolate the blood components and directing the desired analyte to the proper capture zones according to one embodiment of the invention.

FIG. 4 illustrates a perspective view of the main components in the base system according to one embodiment of the invention.

FIG. 5 is a block diagram that illustrates a computing device in the base system according to one embodiment of the invention.

FIG. 6 is a block diagram that illustrates a diagnostic reader module in the computing device according to one embodiment of the invention.

FIG. 7 is a flow chart that illustrates the initial configuration of the base system according to one embodiment of the invention.

FIG. 8 is a flow chart that illustrates the dynamic configuration of base system according to one embodiment of the invention.

### DETAILED DESCRIPTION

### I. Definitions

All publications, patents and patent applications cited herein, whether supra or infra, are hereby incorporated by reference in their entirety.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The term “antibody,” as used herein, includes, but is not limited to a polypeptide substantially encoded by an immunoglobulin gene or immunoglobulin genes, or fragments thereof which specifically bind and recognize an analyte (antigen). “Antibody” also includes, but is not limited to, a polypeptide substantially encoded by an immunoglobulin gene or immunoglobulin genes, or fragments thereof which specifically bind and recognize the antigen-specific binding region (idiotype) of antibodies produced by the host in response to exposure to trichomonas antigen(s). Examples include polyclonal, monoclonal, chimeric, humanized, and single chain antibodies, and the like. Fragments of immunoglobulins, include Fab fragments and fragments produced by an expression library, including phage display. See, e.g., Paul, Fundamental Immunology, 3^{rd} Ed., 1993, Raven Press, New York, for antibody structure and terminology.

The terms “specifically binds to” or “specifically immunoreactive with” refers to a binding reaction which is determinative of the presence of the target analyte in the presence of a heterogeneous population of proteins and other biologics. Thus, under designated assay conditions, the specified binding moieties bind preferentially to a particular target analyte and do not bind in a significant amount to other components present in a test sample. Specific binding to a target analyte under such conditions may require a binding moiety that is selected for its specificity for a particular target analyte. A variety of immunoassay formats may be used to select antibodies specifically immunoreactive with a particular antigen. For example, solid-phase ELISA immunoassays are routinely used to select monoclonal antibodies specifically immunoreactive with an analyte. See Harlow and Lane (1988) Antibodies, A Laboratory Manual, Cold Spring Harbor Publications, New York, for a description of immunoassay formats and conditions that can be used to determine specific immunoreactivity. Typically a specific or selective reaction will provide a signal to noise ratio at least twice background and more typically more than 10 to 100 times background.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like.

As used herein, a “solid support” refers to a solid surface such as a plastic plate, magnetic bead, latex bead, microtiter plate well, glass plate, nylon, agarose, acrylamide, and the like.

“Specific” in reference to the binding of two molecules or a molecule and a complex of molecules refers to the specific recognition of one for the other and the formation of a stable complex as compared to substantially less recognition of other molecules and the lack of formation of stable complexes with such other molecules. Exemplary of specific binding are antibody-antigen interactions, enzyme-substrate interactions, polynucleotide hybridizations and/or formation of duplexes, cellular receptor-ligand interactions, and so forth.

The figures (Figs.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

As used herein any reference to “one embodiment,” “an embodiment,” or “some embodiments” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct physical or electrical contact with each other, but yet still co-operate or interact with each other. The embodiments are not limited in this context.

Also, some embodiments of the invention may be further divided into logical modules. One of ordinary skill in the art will understand that these modules can be implemented in hardware, firmware and/or software. In one embodiment, the modules are implemented in form of computer instructions stored in a computer readable medium when executed by a processor cause the processor to implement the functionality of the module. Additionally, one of ordinary skill in the art will recognize that a computer or another machine with instructions to implement the functionality of one or more logical modules is not a general purpose computer. Instead, the machine is adapted to implement the functionality of a particular module. Moreover, the machine embodiment of the invention physically transforms the electrons representing the images from one state to another in order to attain the desired images.

As used herein, the terms “comprises,” “comprising,” “includes,” “including,” “has,” “having” or any other variation thereof, are intended to cover a non-exclusive inclusion. For example, a process, method, article or apparatus that comprises a list of elements is not necessarily limited to only those elements but may include other elements not expressly listed or inherent to such process, method, article or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

### II. Overview

The invention pertains to a disposable strip that can be used for performing qualitative and quantitative immuno- and chemical assays. On the strip are at least three wells, where the wells can be in fluidic communication with each other via capillary channels. In one well is placed the sample, preferably a liquid sample, for analysis. The sample fluid moves into the other two wells via the capillary channels. One of the wells can serve as a standard that measures the total analyte in the sample. The other well can serve as the reaction well, where individual components of the sample can be identified.

The disposable strip and/or the vial that includes the disposable strip are brought in vicinity of an analyzer (also referred to herein as a “base”) and the analyzer retrieves the necessary information from the strip and/or the vial. The analyzer then configures itself to perform a diagnostic test using a particular disposable strip.

The disposable strip can be placed in an analyzer that detects the individual components of the sample and the total analyte in the sample. The analyzer includes a display system that can display the results of the analysis as well as provide instructions during the operation of the assay.

In one application, the percent total hemoglobin that is hemoglobin Alc (HbAlc) in human red blood cell can be determined. Blood from a subject can be deposited in the sample well. The blood is moved into the other two wells via the capillary channels. In the reference well can be placed a reagent that lyses the cells thereby releasing the hemoglobin from the red blood cells. The concentration of hemoglobin in the reference cell can be measured using infrared or ultraviolet measurements. In the reaction well can be placed a lysate, a known amount of an antibody specific for HbAlc, and a magnetic stirrer. When blood moves into the reaction well, the magnet stirs the liquids in the well thereby mixing them well. The lysate lyses the cells, and the antibody binds to HbAlc. After a specified period of time, the display can instruct the operator to add a diluent to the reaction well. The diluent pushes the liquid in the reaction chamber through another capillary channel towards one or more capture zones. The capture zones have immobilized on them antigens that bind to the bound antibody complex only, and on a separate part of the zone other antigens that bind to all antibodies. The antibody-HbAlc complex can be captured by the antigens in the first part of the capture zone, and all the antibodies can be captured by the antigens in the latter part of the capture zone. A detection system can be used to detect the antibodies bound in the first and the second part of the capture zone. The ratio and/or the sum of the two zones can be used to quantify the amount of HbAlc present in the sample. The ratio of the first zone to the total hemoglobin from the reference cell can provide the percentage of HbAlc in the blood sample. The results can be displayed on the display system

### III. System Overview

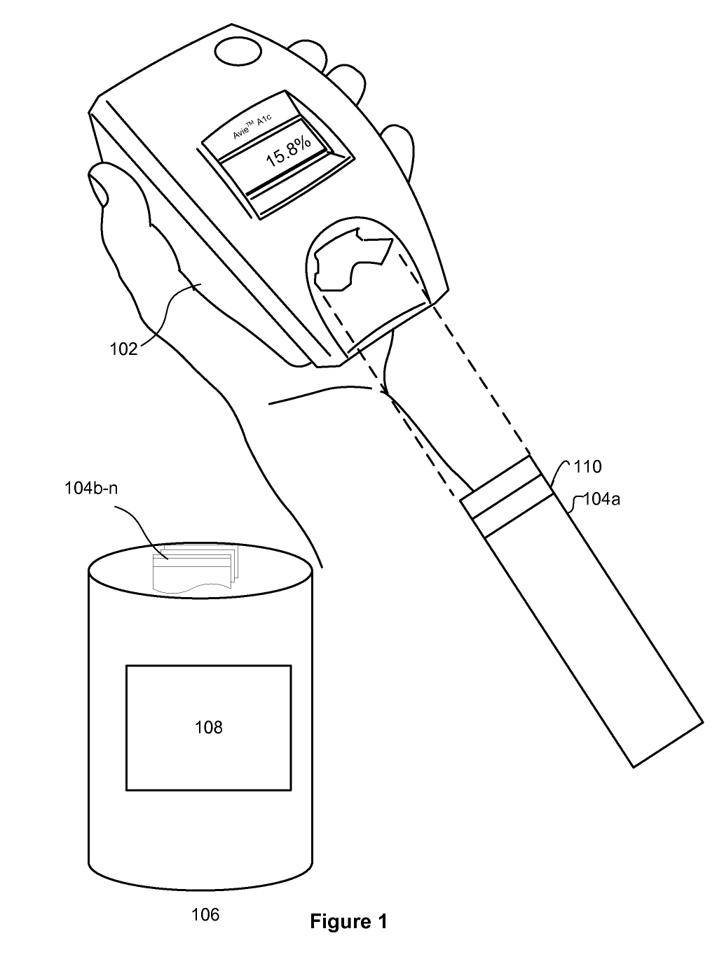

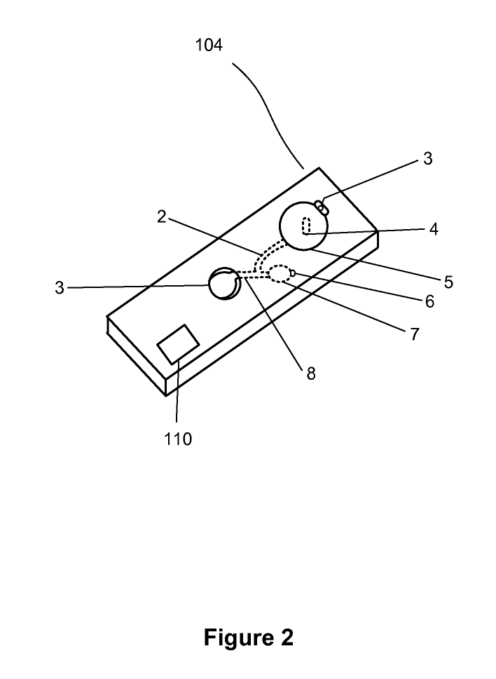

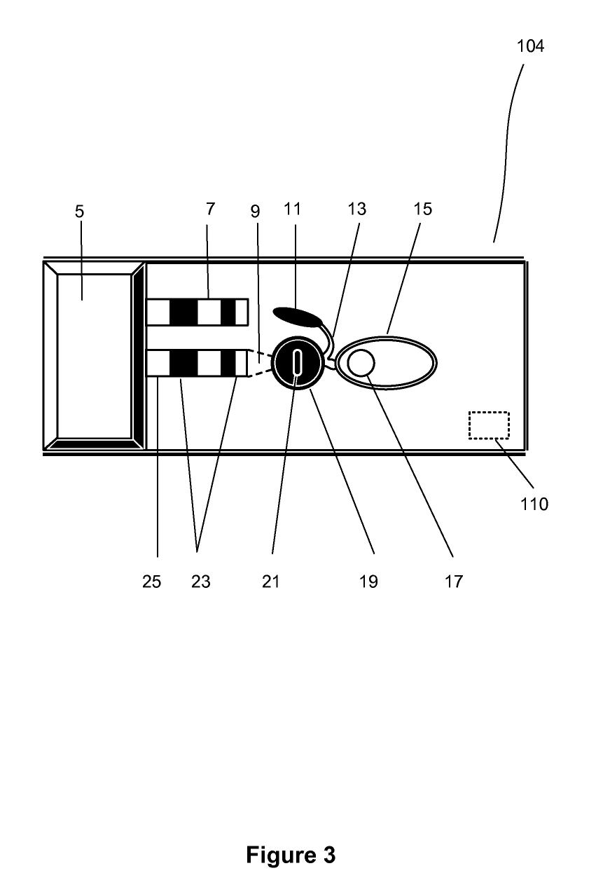

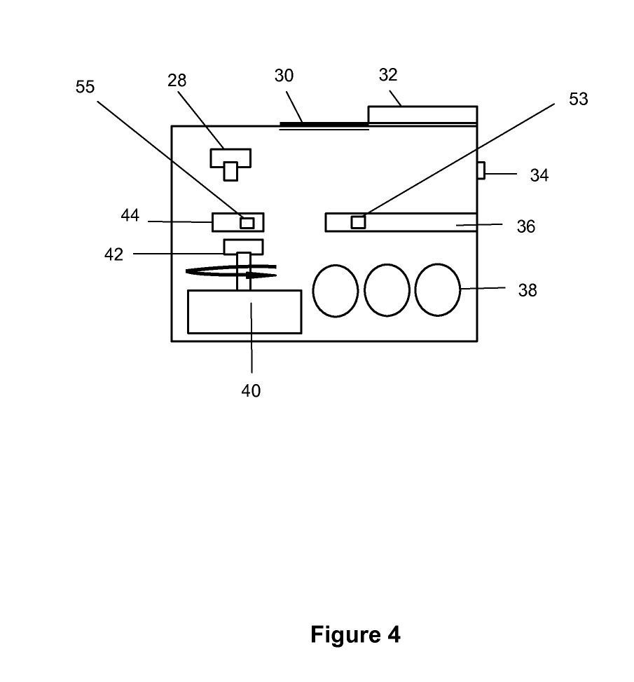

Referring to FIG. 1, the invention provides a vial 106 including one or more disposable strips 104a-n (generically called strip 104), a base 102 and a combination comprising a disposable strip 104 and a base 102. The disposable strip 104 is described below in FIG. 2 and FIG. 3 and the base is described in FIG. 4.

The vial 106 includes vial information module 108 that includes vial information associating the vial 106 with strips 104a-n. This vial information module 108 can be any module capable of storing vial information for later retrieval by a reader. For example, the vial information module 108 can be an integrated circuit capable of storing and transmitting information through a wired or wireless connection, an RFID tag, or a visual tag with encoded information like a color coded tag, a bar code etc. In one embodiment, the vial information includes information regarding association between the vial 106 and strip 104. An example of such association information is a vial identifier that is associated with a strip identifier. This association information can be useful for determining that a strip 104 being used in a test belongs to a particular vial 106. Certain tests can require such a determination and in such cases, the test starts with confirming that the strip 104 being used belongs to a particular vial 106. In another embodiment, the vial information includes the test or an identification of the test to be performed by base 102 using strip 104 from vial 106. In another embodiment, the vial information includes configuration parameters for the base 102. These parameters are specific to various tests performed and/or various strips 104a-n used in the tests. The parameters and the tests are described further below.

The strip 104 includes strip information module 110 that stores information about the strip. The strip information module 110 can be any module capable of storing strip information for later retrieval by a reader. For example, the strip information module 110 can be an integrated circuit capable of storing and transmitting information through a wired or wireless connection, an RFID tag, or a visual tag with encoded information like a color coded tag, a bar code etc. In one embodiment, the strip information in strip information module 110 includes information that associates the strip 104 to vial 106. For example strip information can include an identification that associates the strip 104 to vial 106. In another embodiment, the strip information includes configuration parameters for base 102. In another embodiment, the strip information includes the test or an identification of the test to be performed by base 102 using strip 104 from vial 106.

The base 102 retrieves configuration information either from strip 104 or vial 106. The base 102 can retrieve the information from strip 104 or vial 106 when these objects are brought in proximity to base 102. One of ordinary skill in the art understands that the required proximity varies depending upon the means used to store the information on strip 104 or vial 106. For example, if the information 108, 110 is stored in form of bar codes, the base 102 can use a scanner to read the information when the strip 104 or vial 106 is within few inches or a few feet.

Regardless of how the information is retrieved, the base 102 configures itself using the retrieved information with minimal input from the user. Accordingly, the configuration of base 102 becomes an effortless process that increases test accuracy and reduces possibility of test errors.

One aspect of the disposable strip 104 is illustrated in FIG. 2. The disposable strip 104 can be made by joining together two or more solid supports with grooves present in at least one of the supports. The solid support can be rectangular, circular, oval, or any shape. The support can be made from a suitable material that is selected on its properties, such as good thermal conductivity, clarity for optical transmission, mechanical properties for easy welding, surface properties that allow for uniform coating and stability of reagent, and neutrality to the liquid medium to prevent interference with the assay. For this purpose, suitable plastics include those with high free surface energies and low water sorption, including PETG, polyester (Mylar®), polycarbonate (Lexan®), polyvinyl chloride, polystyrene, SAN, acrylonitrile-butadiene-styrene (ABS), particularly ABS supplied by Borg Warner under the trade name Cyclolac, among others. When the solid support is a hydrophobic plastic, it can be treated by art-known methods to render the surfaces hydrophilic, such as by plasma etching and by corona treatment. Alternatively and equivalently, a commercially-available molded solid support can be used in the practice of the invention.

For purposes of illustration, this embodiment of the invention is described by reference to a disposable strip 104 formed by joining two solid supports. The strip information module 110 can reside on either of the two solid supports or in between. Moreover, at least one of the solid supports has grooves or cavities that serve as the reaction chambers 5 and 7, and capillary channels 2 and 8. The grooves can be any geometric shape, and are preferably circular. The grooves have dimensions that are sufficient volume to hold the samples and to allow for the reaction to occur. Thus, the circular grooves can have a diameter of between about 0.01 mm to about 100 mm, depending on the length and width of the support material, and can have a height of about 0.001 mm to about 4 mm, depending on the thickness of the support material. The diameter and height of the grooves can be easily determined by the one of skill in the art. In one aspect of the invention, one of the support pieces has holes drilled through to the grooves where the holes serve as the vent holes 3 and 6. Further, the holes can allow access to the well where the sample will be placed, such as the sample application well, 1. Prior to the joining of the two pieces, the moveable member, 4, can be inserted in the desired reaction chamber, 5.

An assembled disposable strip is illustrated in FIG. 3, where 5 is an absorbent pad, 7 is an internal reference membrane strip, 9 is a capillary channel, 11 is a reaction chamber, 13 is a capillary channel, 15 is a sample receptacle, 17 is a sample application port, 19 is a reaction chamber, 21 is a moveable member, 23 are capture zones on a base membrane 25 and 110 is the strip information module described above. As apparent from FIGS. 1-3, the strip information module 110 can be placed at various different places on the strip 104 and the placement of strip information module 110 is not meant to be limited by the illustrated placement in FIGS. 1-3. Similarly, the vial information module 108 can be placed in various places on vial 106.

FIG. 4 is a perspective diagram of the main components of the analyzer into which the disposable strip is inserted prior to applying a test sample. The analyzer comprises an electronic display window 30, an input device 28, a heater assembly 44, a motor 40, a magnet 42, a power supply 38, an electronic board 36, a data connector 34 and a reaction chamber 32.

The electronic display window 30 can be, but is not limited to, a Liquid Crystal Display. Input device 28, for example, is a switches or keys on a membrane for function selection from a menu displayed on the LCD. The electronic board 36 comprises a microprocessor and a computing device 53 that controls the operation and mechanics of the analyzer. The power can be supplied by batteries 38, or any other source of alternate electrical power supply. The motor 40 drives the magnet, 42, and the two can be connected, such as by a rod. The defined motion pattern of the motor and magnet are responsible for creating the corresponding motion of the moveable member in one or more of the reaction chambers of the disposable strip. 32, represents a connector to an external power supply and 34 represents a connector for data output.

The disposable strip 104 can be placed on top of a heater assembly 44 that may accommodate a sensor (emitters and or detectors) (not shown) embedded in it or in close proximity but arranged such that a signal goes through the reaction chamber. Another set of sensors that also can serve as emitters, detectors, or both can be positioned on the other side of the disposable strip 104 (not shown). It is to be understood that the reflective beam arrangement, the detector and the emitter can be on the same side of the strip, depending on the detection mechanism that is used. The detection mechanism is not limited to an optical detection method and other methods such as electrical or radioactive detection methods could also be used.

An example of a detection system for automated detection for use with the present disposable strip and associated methods comprises an excitation source, a monochromator (or any device capable of spectrally resolving light components, or a set of narrow band filters) and a detector array. The excitation source can comprise infrared, blue or UV wavelengths and the excitation wavelength can be shorter than the emission wavelength(s) to be detected. The detection system may be: a broadband UV light source, such as a deuterium lamp with a filter in front; the output of a white light source such as a xenon lamp or a deuterium lamp after passing through a monochromator to extract out the desired wavelengths; or any of a number of continuous wave (cw) gas lasers, including but not limited to any of the Argon Ion laser lines (457, 488, 514, etc. nm) or a HeCd laser; solid-state diode lasers in the blue such as GaN and GaAs (doubled) based lasers or the doubled or tripled output of YAG or YLF based lasers; or any of the pulsed lasers with output in the blue.

The emitted light from the sample or the reactants in the reaction well can be detected with a device that provides spectral information for the substrate, e.g., a grating spectrometer, prism spectrometer, imaging spectrometer, or the like, or use of interference (bandpass) filters. Using a two-dimensional area imager such as a CCD camera, many objects may be imaged simultaneously. Spectral information can be generated by collecting more than one image via different bandpass, longpass, or shortpass filters (interference filters, or electronically tunable filters are appropriate). More than one imager may be used to gather data simultaneously through dedicated filters, or the filter may be changed in front of a single imager. Imaging based systems, like the Biometric Imaging system, scan a surface to find fluorescent signals.

Other embodiments appropriate for this system include the use of reagent-coated membranes systems as part of the strip positioned in a way that allows continuity and directed sample flow within the entire strip system. The sensory systems would be positioned to be capable of monitoring the membrane portions of the strip for the analyte or responses being tested for.

The heater assembly 44 also includes an information sensor 55 that is configured to read vial information and/or strip information from vial 106 and/or strip 104. In one embodiment, information sensor 55 is an RFID tag reader that retrieves the RFID information from one or more RFID tags 110, 108 on strip 104 or vial 106. In another embodiment, information sensor 55 is an imaging sensor that retrieves the encoded information from visual tags on strip 104 or vial 106. In yet another embodiment, the information sensor 55 is an integrated circuit capable of retrieving information from integrated circuit 110, 108 on strip 104 or vial 106.

The information sensor 55 is communicatively coupled to computing device 53 on electronic board 36. The information sensor 55 transmits the retrieved information to computing device 53 for further processing as described below. In one embodiment, the functionality of the information sensor 55 is included in one or more sensors in heater assembly 44 described above and therefore a separate information sensor 55 does not exist on base 102.

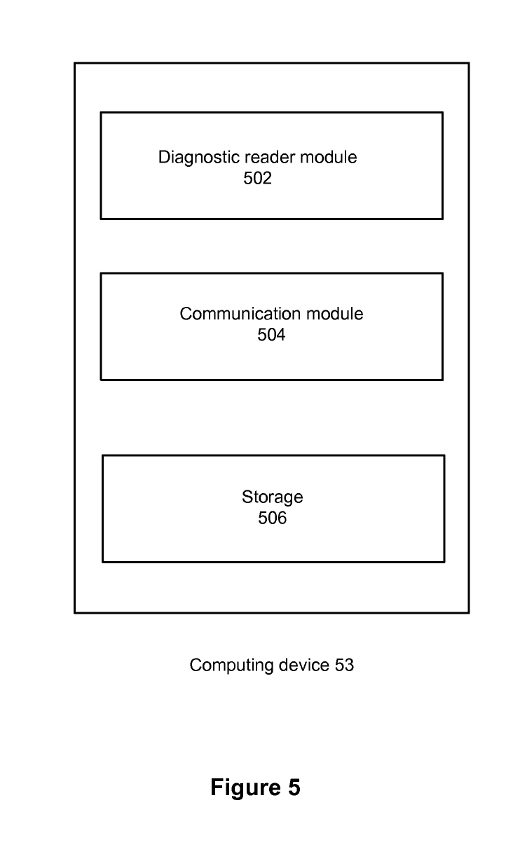

The computing device 53 receives the information from information sensor 55 and configures the base 102 based on the received information. In one embodiment, the functionality of computing device 53 is performed by the microprocessor on the electronic board 36 and the computing device 53 does not exist as a separate entity. The computing device 53 is described below in FIG. 5.

FIG. 5 is a block diagram that illustrates the computing device 53. The computing device 53 includes a diagnostic reader module 502, a communication module 504 and storage 506.

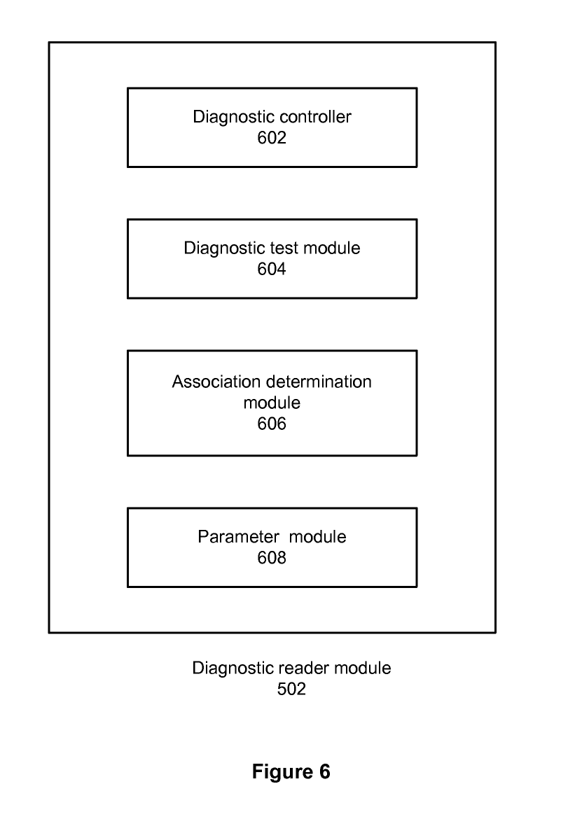

The diagnostic reader module 502 is communicatively coupled to communication module 504 and storage 506, and the diagnostic reader module 502 is configured to retrieve test algorithms and various parameters from storage 506. The diagnostic reader module 502 uses the retrieved values to initialize base 102. The diagnostic reader module 502 also receives information from information sensor 55 through communication module 504 and dynamically configures base 102 to perform certain tests with certain parameters for a particular strip 104. The diagnostic reader module 502 is described below in FIG. 6.

The communication module 504 is communicatively coupled to information sensor 55 and diagnostic reader module 502. The communication module 504 receives information from information sensor 55 and transmits the received information to diagnostic reader module 502.

The storage 506 is communicatively coupled to diagnostic reader module 502 and storage 506 stores various parameters for one or more test algorithms. In one embodiment, storage 506 also stores the one or more test algorithms corresponding to a particular vial 106 or strip 104. In another embodiment, storage 506 also stores test algorithm identification corresponding to the test algorithm. This information is used to configure base 102 with a particular test algorithm.

In one embodiment, the storage 506 also stores a list of vial identifications for various vials 106 and a list of strip identifications corresponding to each vial 106. These association lists are used to confirm that a strip 104 is not mistakenly placed into an incorrect vial 106. In one embodiment, storage 506 is communicatively coupled to a database (not shown) and the data in storage 506 is updated through the database. In another embodiment, storage 506 is communicatively coupled to a client device (not shown) and the data in storage 506 is updated through the client device.

FIG. 6 is a block diagram that illustrates the diagnostic reader module 502 according to one embodiment of the invention. The diagnostic reader module 502 comprises a diagnostic controller 602, a diagnostic test module 604, a parameter module 608 and an association determination module 606.

The diagnostic controller 602 is configured to signal other modules in diagnostic reader module 502 to perform their respective tasks at appropriate times. The diagnostic controller 602 is communicatively coupled to diagnostic test module 604, parameter module 608 and association determination module 606. The diagnostic controller 602 is also communicatively coupled to information sensor 55 for receiving information retrieved by information sensor 55.

The diagnostic test module 604 is communicatively coupled to diagnostic controller 602, storage 506 and the microprocessor on electronic board 36. The diagnostic test module 604 receives the identification information for vial 106 or strip 104 from diagnostic controller 602 and determines the diagnostic test to be performed by base 102. In one embodiment determining the diagnostic test comprises retrieving from storage 506 a test corresponding to the received identification information for vial 106 or strip 104. In another embodiment, the diagnostic test module 604 receives an identification for the test from vial 106 or strip 104 through diagnostic controller 606. In this embodiment, determining the diagnostic test comprises retrieving from storage 506 a test corresponding to the received test identification. In yet another embodiment, the diagnostic test module 604 receives the test itself from vial 106 or strip 104 and determining the test comprises receiving the test.

Regardless of how the diagnostic test module 604 determines the test, the diagnostic test module 604 next configures base 102 with the determined diagnostic test. Configuring the base 102 with a diagnostic test comprises signaling the microprocessor on electronic board 36 to perform the determined test. An example of diagnostic test includes a single use electronic assay described in U.S. Pat. No. 5,580,794. Other examples include tests for measuring glucose, urea nitrogen, hemoglobin, or blood components. An example of such tests is described in U.S. Pat. No. 4,627,445. Another example of diagnostic tests includes a test for measuring clotting time of blood as described in U.S. Pat. No. 4,197,734. Additional examples of diagnostic tests will be readily apparent to one of ordinary skill in the art.

The association determination module 606 is communicatively coupled to the diagnostic controller 602 and storage 506. The association determination module 606 receives the identification information for vial 106 and strip 104 from diagnostic controller 602. The association determination module 606 then searches the list of associations in storage 506 and determines if received information for vial 106 and strip 104 is associated with each other.

In one embodiment, the association determination module 606 does not determine the association between vial 106 and strip 104 by querying the association lists in storage 506. Instead, the association determination module 606 receives the association information from vial 106 or strip 104. The information sensor 55 retrieves from strip 104 one or more vial identifiers associated with the strip 104 or retrieves from vial 106 identifiers for one or more strips 104 associated with vial 106. The information sensor 55 transmits the retrieved information to association determination module 606 through diagnostic controller 606. The association determination module 606 uses this received information to determine whether vial 106 and strip 104 are associated with each other. Regardless of how the association determination module 606 determines the association between the vial 106 and strip 104, the association determination module 606 transmits the results of the determination to diagnostic controller 602.

The parameter module 608 is communicatively coupled to diagnostic controller 602 and storage 506. In one embodiment, the parameter module 604 receives the identification information for vial 106 or strip 104 from diagnostic controller 602 and retrieves from storage 506 the parameter values corresponding to the received information. In another embodiment, the parameter module 608 receives the parameters from vial 106 or strip 104 through diagnostic controller 602. Regardless of how the parameter module 604 receives the parameters, the parameter module 604 next configures base 102 with the determined parameter values. Configuring the base 102 with parameter values for a diagnostic test comprises signaling the microprocessor on electronic board 36 to use the determined parameter values while performing the diagnostic test. Examples of parameters include type of detectable moiety or label on the strip, absorption and emission maxima for the label and the calibrators or standards for the labels. These examples are discussed further below. Other parameters for other tests are readily apparent to one of ordinary skill in the art.

### VI. Analyzer Configuration

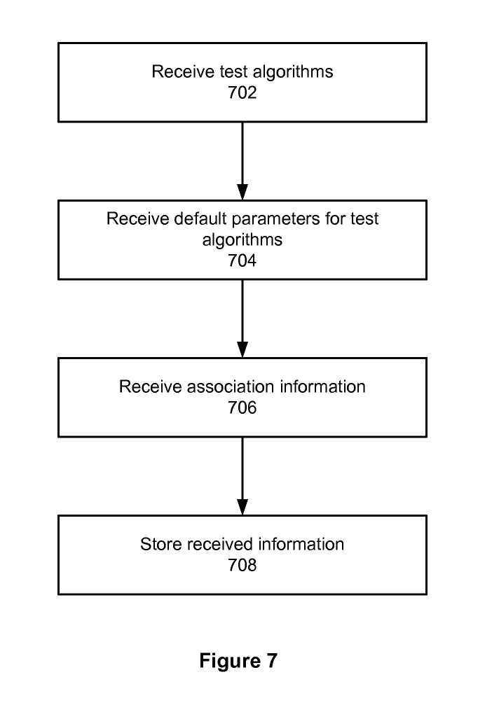

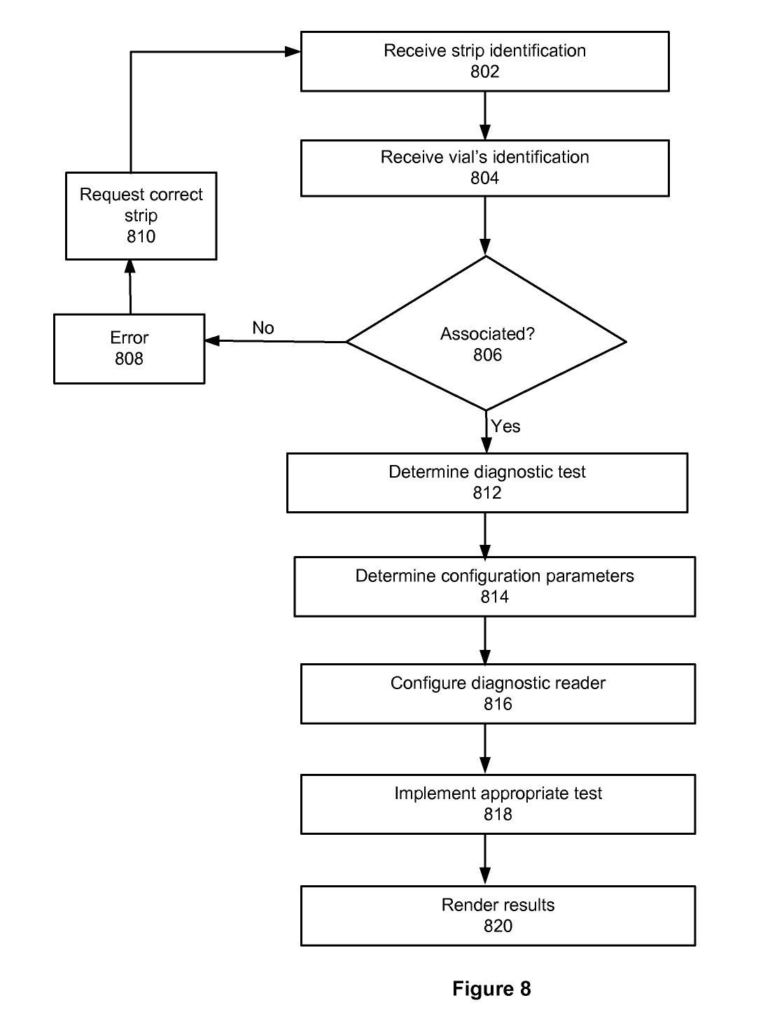

FIG. 7 is a flow chart that illustrates the initial configuration of base 102 according to one embodiment of the invention. As discussed above, storage 506 is communicatively coupled to a database (not shown) or a client device (not shown). The storage 506 receives 702 test algorithms from the client device or the database. The storage 506 also receives 704 default parameters for the test algorithms. These parameters are used to initialize the test and one or more of these parameters are dynamically updated according to the information received from strip 104 or vial 106. This dynamic update is described below in FIG. 8.

Next, the storage 506 receives 706 association information from the client device or database. The association information links a strip 104 to vial 106. After receiving the association information, storage 506 stores 708 the received test algorithms, default parameters and association information. In one embodiment, storage 506 stores the information as it receives the information. Accordingly, storage 506 receives and stores test algorithms, then receives and stores default parameters, and next receives and stores the association information.

FIG. 8 is a flow chart that illustrates a method for dynamically configuring base 102 and implementing an appropriate test according to one embodiment of the invention. Strip 104 is brought in vicinity of base 102 and information sensor 55 reads the identification information from strip information module 110. In one embodiment, the information sensor 55 additionally reads the configuration test and/or one or more configuration parameters from strip information module 110. The information sensor 55 then retrieves the vial's identification information from vial information module 108. In one embodiment, the information sensor 55 additionally reads the configuration test and/or one or more configuration parameters from vial information module 108. One of ordinary skill in the art will understand that configuration test and/or one or more configurations parameters can be retrieved either from strip information module 110 or vial information module 108. Additionally, the information sensor 55 can retrieve one or more parameters and test partly or wholly from either strip information module 110 or vial information module 108. In one embodiment, the information sensor 55 also retrieves from vial information module 108 identifiers for one or more strips 104 associated with vial 106. In another embodiment, information sensor 55 retrieves from strip identification module 110 identifiers for one or more vials 106 associated with strip 104. After retrieving the information, the information sensor 55 transmits the retrieved information to diagnostic controller 602. The diagnostic controller 602 in turn transmits the information to association determination module 606.

The association determination module 606 receives 802, 804 the strip identification information and vial identification information. The association determination module 606 then queries the association lists in storage 506 and determines 806 if the received strip identification information is associated with received vial identification information. Alternatively, the association determination module 606 receives strip identifiers associated with vial 106 or vial identifiers associated with strip 104 and determines from these associated identifiers if the received strip identifier information is associated with the received vial identifier information. If not, the association determination module 606 transmits 808 an error to the microprocessor on electronic board 36. The microprocessor then signals 810 the electronic display window 30 to display an error message and request a strip 104 associated with vial 106. The electronic display window 30 in one of many ways of rendering the error message. Other means of rendering an error message include an audio or a visual presentation that informs the operator regarding the lack of association between vial 106 and strip 104.

After the base 102 receives another strip 104 from the operator, steps 802-810 are repeated until a strip 104 associated with vial 106 is received. Accordingly, steps 802-810 describe a method for determining whether a strip 104 is associated with a particular vial 106. In one embodiment, base 102 does not make this association determination and steps 806-810 are skipped.

Regardless of whether the association determination is performed or not, the diagnostic controller 602 next signals the diagnostic test module 604 and diagnostic test module 604 determines 812 the test associated with the received strip identification information and/or vial identification information. In one embodiment, the diagnostic test module 604 receives the strip identification information and/or the vial identification information from information sensor 55 and determines the test stored in storage 506 that is associated with the received identification. In another embodiment, the diagnostic test module 604 receives an identification for the diagnostic test from information sensor 55 and retrieves the test corresponding to the test identification from storage 506. In yet another embodiment, the diagnostic test module 604 receives the diagnostic test itself from information sensor 55. In yet another embodiment, base 102 is pre-configured with a test and accordingly diagnostic test module 604 does not determine or receive a test.

Regardless of whether the diagnostic test is determined or not, the diagnostic controller 602 next signals the parameter module 608 and the parameter module 608 determines 814 the configuration parameters for base 102. In one embodiment, the parameter module 608 receives strip identification and/or vial identification from information sensor 55 and queries the list in storage 506 to determine the configuration parameters corresponding to the received identification information. In another embodiment, the parameter module 608 receives the configuration parameters from information sensor 55.

Next, the diagnostic controller 602 configures 816 base 102 with the determined parameters and optionally the determined test. The base 102 then implements 818 the test using the new configuration and renders 820 the results for the operator.

### V. Operation

A general mode of operation of the device shown in FIG. 3 involves the insertion of the disposable strip shown in FIGS. 1 and 2 into a receptacle that allows the strip on one position only. For an assay with a specific temperature requirement, the heater assembly, 44, heats the disposable strip to the desired temperature controlled by the microprocessor. The LCD prompts simple steps, after the strip is inserted and the analyzer turned on, which the operator can follow including the addition of the sample to the sample receptacle. Optionally, the instrument can also have sensors to determine the presence of adequate amounts of sample in the reaction chambers and a mechanism to initiate and stop the timing of the assay. The sensor detects the signals from the completion of the reaction, such as measuring the transmission of an optical signal emitted and directed through the walls of the reaction chamber.

The applied sample is accurately distributed into the various reaction chambers via the capillary channels. The positioning of the reaction chambers can be such that independent reactions can occur in the various reaction chambers even though they share a common sample from the same pool. The defined modes of movement of the moveable member ensures proper mixing of the reagent and sample mixtures and also contributes in inter chamber reagent and sample interchange. For assays that require quantification of an analyte, the sensory system monitors the changes either in one or more reaction chambers, a membrane system or the moveable members, until the desired end point is achieved. For assays requiring just the determination of the presence of an analyte, the sensory system monitors the specific parts of the strip for the appropriate duration of time. The microprocessor computes the results quantitatively or qualitatively according to the determined test and/or parameters and the results are displayed on the LCD. The strip can then be removed at the end of the assay and disposed.

Thus, the operator inserts the strip in an analyzer that configures itself according to the information on the inserted strip and/or the information on the vial of the inserted strip. The analyzer then receives a sample, analyzes the sample and then displays the results, typically within a few minutes or seconds, depending on the assay type.

### VI. Detection of Hemoglobin Alc (HbAlc)

Glycated hemoglobin refers to a series of minor hemoglobin components that are formed through the attachment of glucose to the hemoglobin molecule. The human red blood cell is freely permeable to glucose. Within each red blood cell, glycated hemoglobin is formed at a rate that is directly proportional to the ambient glucose concentration. Approximately 97% of the total hemoglobin in circulating red blood cells is hemoglobin A. Hemoglobin A consists of four polypeptide chains, two a-chains and two b-chains. Glycation of the Hemoglobin A occurs through the covalent coupling of glucose with the N-terminal valine amino acid of each b-peptide chain. An unstable Schiff base (aldimine) is initially formed which then undergoes an irreversible Amadori rearrangement to form a stable ketoamine, Hemoglobin Alc (HbAlc).

The life-span of hemoglobin A containing red blood cells averages 120 days. The percentage of Hemoglobin A that is glycated to HbAlc is directly proportional to the time that red blood cells are exposed to glucose and to the average glucose concentration encountered. Measurement of the HbAlc fraction gives an integrated picture of the average blood glucose concentration during the half-life of the red cells, that is, over the last 60 days. The level of HbAlc is usually expressed as a percentage of total hemoglobin.

In normal subjects, HbAlc is typically in the range 3-6% of total hemoglobin. In patients with elevated glucose levels e.g. in the case of Type 1 and Type 2 diabetes, the level may rise to twice the upper limit of normal or more.

Long-term control of glucose levels in diabetics is very important. Too much glucose in the blood over many years can damage the eyes, kidneys and nerves. It also increases the risk for heart and blood vessel disease. The measurement of HbAlc as a percentage of total hemoglobin provides a valuable means of assessing the long-term control of glucose levels and also constitutes an important risk indicator for identifying Type 1 and Type 2 diabetics.

A sample of blood from a subject can be obtained in deposited in the sample well 15 of the disposable strip (FIG. 2). The blood moves to the reaction chambers 11 and 19 via the capillary channels 13. The reaction chamber 11 can serve as the reference where the total hemoglobin is measured. The reaction chamber 19 measures the HbAlc in the blood sample. The ratio of HbAlc to the total hemoglobin provides the percentage of total hemoglobin that is HbAlc.

Both of the reaction chambers contain a lysing agent. The lysing agent lyses the whole blood samples thereby releasing the hemoglobin. The lysing agents are typically surfactants, and preferably nonionic surfactants, such as for example TRITON™ X-100. The reaction chamber 19 additionally contains an antibody that can detect HbAlc. The antibody can be a monoclonal or polyclonal antibody (Ab), or Ab fragment containing the antigen binding site, or complementarity determining region (CDR), such as an F(ab′)₂ or Fab fragment. The detectable moiety or label may be a radioactive, fluorescent or chemiluminescent substance, or an enzyme. Alternatively, a labeled-second Ab which recognizes the species specific Fc fragment of the first Ab may also be used. Further, the antibody may be labeled with a detectable label.

In one aspect, the detectable label is a fluorescent molecule. Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester), rhodamine (5,6-tetramethyl rhodamine), substituted rhodamine compounds, and the cyanine dyes Cy3, Cy3.5, Cy5, Cy5.5 and Cy7. The absorption and emission maxima, respectively, for these fluorophores are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio. As another alternative, in place of an added label, the bound hemoglobin itself, due to its peroxidase-like properties, can generate a detectable signal. This is accomplished by adding hydrogen peroxide, with or without addition of another substrate (e.g. isoluminol).

The cells in the two reaction wells are lysed. In the reference well 11, the total amount of hemoglobin can be obtained by spectroscopic methods, such as measuring in the UV region or the infrared region. The spectroscopic apparatus is known in the art and is incorporated within the portable handheld machine. In particular, the measurements can be made at 880 nm and at 580 nm. In the reaction well 19, the antibodies bind to HbAlc. In order to ensure complete reaction, the liquids in the reaction wells can be magnetically stirred, and optionally heated to a higher temperature.

Upon completion of the reaction, a diluent can be added to the sample well 15. The diluent causes the reactants in 19 to move through the capillary channels 9 towards the capture zones 23. The capture zones can be antigens or other compounds that can specifically bind to the antibody-HbAlc complex, any antibody, and the like or combinations thereof. Thus, in one aspect, the first capture zone contains antigens that specifically bind to the antibody-HbAlc complex, while the second capture zone contains antigens that bind to the antibody and the antibody-HbAlc complex. The absorbent pad 5 absorbs all the liquid and can help in drawing the liquid from the wells through the membranes.

The amount of material in each capture zone can be determined by using the detection systems described above. Calibrators or standards that are run with the assay provide calibration (or standard) curves from which the % HbAlc in the sample is determined using the measured signal. The sum of all the capture zones preferably equals the amount of antibody that was placed in the reaction well, and can provide an internal control to determining the percentage of reaction that has occurred. The concentration of the antibody-HbAlc complex can be determined from the reading of the first capture zone. The % HbAlc in the blood sample can be determined by dividing the concentration of the antibody-HbAlc complex with the total concentration of hemoglobin.

In another aspect, a reference membrane 7 (FIG. 2) can be included in the disposable strip. The reference membrane can have deposited upon it known concentrations of the antigen-antibody-HbAlc complex, and antigen-antibody complex. The spectrophotometric measurements form the reference membrane can be used to calibrate the readings from the active membrane 25.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

1. A configurable analyzer for implementing a test, the configurable analyzer comprising:
a diagnostic controller for determining an identification associated with one or more objects from a lists consisting of one of a plurality of strips from a vial and the vial;
a parameter module, communicatively coupled to the diagnostic controller, for determining a configuration parameter for the test based on the determined identification and for configuring the analyzer to perform the test based on the determined parameter.

2. The analyzer of claim 1 wherein
the determined identification is associated with a strip from the plurality of strips, and
the diagnostic controller is further configured to determine a vial identification associated with the vial, and
the analyzer further comprises:
an association determination module, communicatively coupled to the diagnostic controller, for determining whether the vial identification is associated with the identification; and
a rendering device for rendering an error if the vial identification is not associated with the identification.

3. The analyzer of claim 2 wherein:
the diagnostic controller is configured to receive a plurality of strip identifications corresponding to the plurality of strips from the vial; and
the association determination module is configured to determine whether the identification corresponds to one of the received plurality of strip identifications.

4. The analyzer of claim 2 further comprising:
a storage, communicatively coupled to the diagnostic controller, for storing a plurality of vial identifications and a plurality of strip identifications corresponding to each of the stored vial identification;
wherein the association determination module is configured to determine from the stored plurality of vial identifications and the plurality of strip identifications whether the identification corresponds to the determined vial identification.

5. The analyzer of claim 1 further comprising:
a diagnostic test module, communicatively couple to the diagnostic controller, for determining the test based on the determined identification and configuring the analyzer based on the determined test.

6. The analyzer of claim 1 further comprising:
a diagnostic test module, communicatively couple to the diagnostic controller, for receiving the test from the strip or the vial and configuring the analyzer based on the received test.

7. The analyzer of claim 1 further comprising:
a diagnostic test module, communicatively couple to the diagnostic controller, for
receiving a test identification form the strip or the vial,
determining the test based on the received test identification, and
configuring the test based on the determined test.

8. The analyzer of claim 1 wherein
the parameter module determines the configuration parameter for the test based on the determined identification by receiving the configuration parameter from the one or more object associated with the determined identification.

9. A method for configuring an analyzer, the method comprising:
determining an identification associated with one or more objects from a lists consisting of one of a plurality of strips from a vial and the vial;
determining a configuration parameter for a test based on the determined identification;
configuring the analyzer to perform the test based on the determined parameter; and
performing the test.

10. The method of claim 9 wherein the determined identification is associated with a strip from the plurality of strips, the method further comprising:
determining a vial identification associated with the vial;
determining whether the vial identification is associated with the identification; and
rendering an error if the vial identification is not associated with the identification.

11. The method of claim 10 wherein determining the association between the identification and the vial identification comprises:
receiving a plurality of strip identifications corresponding to the plurality of strips from the vial; and
determining whether the identification corresponds to one of the received plurality of strip identifications.

12. The method of claim 10 wherein determining the association between the identification and the vial identification comprises:
storing a plurality of vial identifications and a plurality of strip identifications corresponding to each of the stored vial identification; and
determining from the stored plurality of vial identifications and the plurality of strip identifications whether the identification corresponds to the determined vial identification.

13. The method of claim 9 further comprising:
determining the test based on the determined identification; and
configuring the analyzer based on the determined test.

14. The method of claim 9 further comprising:
receiving the test from the strip or the vial; and
configuring the analyzer based on the received test.

15. The method of claim 9 further comprising:
receiving a test identification form the strip or the vial;
determining the test based on the received test identification; and
configuring the analyzer based on the determined test.

16. The method of claim 9 wherein determining the configuration parameter for the test based on the determined identification comprises receiving the configuration parameter from the one or more object associated with the determined identification.
